(19) United States
(12) Patent Application Publication
(10) Pub. No.: US 2019/0015044 A1
(43) Pub. Date: Jan. 17, 2019

(54) PORTABLE ELECTRONIC DEVICE AND PORTABLE ELECTRONIC DEVICE STRAP

(71) Applicant: SEIKO EPSON CORPORATION, Tokyo (JP)

(72) Inventors: Mitsuto YANAGISAWA, Matsumoto-shi (JP); Ippei HONMA, Shiojiri-shi (JP)

(73) Assignee: SEIKO EPSON CORPORATION, Tokyo (JP)

(21) Appl. No.: 16/025,604

(22) Filed: Jul. 2, 2018

(30) Foreign Application Priority Data
Jul. 14, 2017 (JP) 2017-137779

(51) Int. Cl. A61B 5/00; A61B 5/0205; A44C 5/00

(52) U.S. Cl. CPC A61B 5/681; A61B 5/02416; A44C 5/0053; A61B 5/0205

(57) ABSTRACT

A portable electronic device includes: a detection unit which detects at least one of biological information and motion information of a subject; a case which accommodates the detection unit; and a strap which fixes the case to the subject. The strap has a first surface which faces the subject when the case is fixed to the subject. The first surface is provided with a plurality of first protrusions, satisfying 0.03 mm≤D≤1.0 mm and 1<P/D≤3, where P/D is a ratio of a pitch P representing a distance between centers of two of the first protrusions and an outer dimension D of each of the first protrusions.

### BACKGROUND

### 1. Technical Field

The present invention relates to a portable electronic device and a portable electronic device strap.

### 2. Related Art

Recently, with improvements in sensing technology, various manufacturers have been producing and selling wristwatch-type activity trackers which measure biological information and motion information, as an example of a portable electronic device. For example, JP-A-2010-110634 discloses a wristwatch-type electronic device worn to be used at a wearing part (subject) such as the wrist with a strap or the like. With such a wristwatch-type activity tracker, its case needs to be in tight contact with the wearing part to measure biological information stably. Also, to achieve continuous measurement over a long period, the wristwatch-type activity tracker needs to be worn continuously and comfortably with little burden on the wearer.

To cope with the need to achieve comfortable wearing with little burden on the wearer in order to achieve continuous measurement over a long period as described above, for example, JP-A-2009-249560 discloses using a strap made up of a silicone material having tackiness whose surface is at least mixed or coated with a powder.

However, when a wristwatch-type activity tracker is worn using the strap made up of a silicone material whose surface is at least mixed or coated with a powder, disclosed in JP-A-2009-249560, abrasion and friction or the like in use may scrape off (separate) the powder or the like on the strap surface. This makes it difficult to continue comfortable wearing with reduced burden on the wearer. In other words, there is the problem of an unfavorable feeling of use to the wearer.

### SUMMARY

An advantage of some aspects of the invention is to solve at least a part of the problems described above, and the invention can be implemented as the following forms or application examples.

### Application Example 1

A portable electronic device according to this application example includes: a detection unit which detects at least one of biological information and motion information of a subject; a case which accommodates the detection unit; and a strap which fixes the case to the subject. The strap has a first surface which faces the subject when the case is fixed to the subject. The first surface is provided with a plurality of first protrusions, satisfying 0.03 mm≤D≤1.0 mm and 1<P/D≤3, where P/D is a ratio of a pitch P representing a distance between centers of two of the first protrusions and an outer dimension D of each of the first protrusions.

With the portable electronic device according to this application example, the plurality of first protrusions, arranged on the first surface of the strap facing the subject side when the case is fixed to the subject and satisfying the foregoing condition about the relation between the pitch P and the outer dimension D, can secure a proper spacing between the subject and the strap. Also, a proper size and strength of the first protrusions can be secured, thus making the first protrusions less likely to be separated due to abrasion or friction. Thus, even if the portable electronic device is used continuously for a long period with the case in tight contact with the wearing part, comfortable wearing can continue with reduced burden on the wearer.

### Application Example 2

In the portable electronic device according to the application example, it is preferable that the first protrusions are arranged, satisfying 0.03 mm≤D≤0.8 mm and 1.4<P/D≤3.

According to this application example, the plurality of first protrusions, arranged on the first surface of the strap facing the subject side when the case is fixed to the subject and satisfying the condition of this application example about the relation between the pitch P and the outer dimension D, can achieve more comfortable wearing with reduced burden on the wearer.

### Application Example 3

In the portable electronic device according to the application example, it is preferable that 0.005 mm≤d≤0.2 mm is satisfied, where a distance from a top part of the first protrusions to the first surface is defined as a depth d.

According to this application example, a reduction in the strength of the first protrusions can be prevented. This can make the first protrusions less likely to be separated from the strap due to abrasion or friction and thus can improve the durability of the strap part.

### Application Example 4

In the portable electronic device according to the application example, it is preferable that the pitch P is 0.005 mm≤P≤0.3 mm.

According to this application example, the first protrusions are formed with a smaller pitch than a fingerprint (palm print), whose pitch is considered to be 0.3 mm to 0.5 mm. Therefore, for example, when the strap is placed on the wrist, the fingerprint and the first protrusions are not fitted with each other. This makes the first surface of the strap less likely to come into direct contact with the skin and thus can maintain a good feeling to the touch.

### Application Example 5

In the portable electronic device according to the application example, it is preferable that the first protrusions have at least a first line in which the first protrusions are arranged in a first direction and a second line in which the first protrusions are arranged in the first direction, with a predetermined spacing from the first line in a second direction orthogonal to the first direction, and that the first protrusions in the first line and the second line are arranged with a shift half the pitch P in the first direction.

According to this application example, the first protrusions in the first line and the second line are arranged with a shift half the pitch P in the first direction. Therefore, an effective space to discharge moisture, for example, sweat, can be secured between the first protrusions next to each other. Narrowing the spacing between the first line and the second line can increase the number of lines where the first protrusions are arranged. That is, the number of first protrusions arranged can be increased. This can achieve a good feeling to the touch.

### Application Example 6

In the portable electronic device according to the application example, it is preferable that the strap includes a case fitting section and a strap main body extending from the case fitting section, and that the first protrusions are provided on the strap main body.

According to this application example, a part of the case fitting section close to the case can come into tight contact with the subject, whereas the strap main body having a large contact area can be prevented from coming into tight contact with the subject by the first protrusions provided thereon. This can achieve secure tight contact of the case and a good feeling to the touch of the strap.

### Application Example 7

In the portable electronic device according to the application example, it is preferable that the strap has a second surface which faces opposite to the subject side when the case is fixed to the subject, that the second surface is provided with a plurality of second protrusions, and that the second protrusions have a pitch P2 and an outer dimension D2 that are different from those of the first protrusions.

According to this application example, the plurality of second protrusions provided on the second surface are provided with the pitch P2 and the outer dimension D2, which are different from those of the first protrusions. Thus, the adhesion of fluff and dirt due to contact with the external environment such as clothes, which tends to occur on the second surface situated on the surface side opposite to the subject side, can be made less likely to occur.

### Application Example 8

In the portable electronic device according to the application example, it is preferable that the first surface and the second surface are made up of different materials from each other.

According to this application example, for example, the first surface and the second surface can be made different from each other in softness or color. For example, if the second surface is made up of a harder material than the first surface, the strength of the second surface increases. This can increase the strength of the strap while maintaining a good feeling of wearing of the first surface. Meanwhile, for example, if the second surface is in a color that makes dirt less visible than on the first surface, a portable electronic device that has better design and achieves a good feeling of wearing can be provided.

### Application Example 9

A portable electronic device strap according to this application example is a portable electronic device strap which fixes a case to a subject, the case accommodating a detection unit which detects at least one of biological information and motion information of the subject. The portable electronic device strap includes: a case fitting section to be fixed to the case; a strap main body extending from the case fitting section; and a first surface which faces the subject of the strap main body when the case is fixed to the subject. The first surface is provided with a plurality of first protrusions, satisfying 0.03 mm≤D≤1.0 mm and 1<P/D≤3, where P/D is a ratio of a pitch P representing a distance between centers of two of the first protrusions and an outer dimension D of each of the first protrusions.

With the portable electronic device strap according to this application example, the plurality of first protrusions, arranged on the first surface of the strap main body facing the subject side when the case is fixed to the subject using such a strap, and satisfying the foregoing condition about the relation between the pitch P and the outer dimension D, can secure a proper spacing between the subject and the strap main body. Also, a proper size and strength of the first protrusions can be secured, thus making the first protrusions less likely to be separated due to abrasion or friction. Thus, even if the portable electronic device is used continuously for a long period with the case in tight contact with the wearing part, comfortable wearing can continue with reduced burden on the wearer.

### Application Example 10

In the portable electronic device strap according to the application example, it is preferable that the first protrusions are arranged, satisfying 0.03 mm≤D≤0.8 mm and 1.4<P/D≤3.

According to this application example, the plurality of first protrusions, arranged on the first surface of the strap main body facing the subject side and satisfying the condition of this application example about the relation between the pitch P and the outer dimension D, can achieve more comfortable wearing with reduced burden on the wearer.

### Application Example 11

In the portable electronic device strap according to the application example, it is preferable that 0.005 mm≤d≤0.2 mm is satisfied, where a distance from a top part of the first protrusions to the first surface is defined as a depth d.

According to this application example, a reduction in the strength of the first protrusions can be prevented. This can make the first protrusions less likely to be separated from the strap main body due to abrasion or friction.

### Application Example 12

In the portable electronic device strap according to the application example, it is preferable that the pitch P is 0.005 mm≤P≤0.3 mm.

According to this application example, the first protrusions are formed with a smaller pitch than a fingerprint (palm print), whose pitch is considered to be 0.3 mm to 0.5 mm. Therefore, for example, when the case fitting section and the strap main body are placed on the wrist, the fingerprint and the first protrusions are not fitted with each other. This makes the first surface of the strap main body less likely to come into direct contact with the skin and thus can maintain a good feeling to the touch.

### Application Example 13

In the portable electronic device strap according to the application example, it is preferable that the first protrusions have at least a first line in which the first protrusions are arranged in a first direction and a second line in which the first protrusions are arranged in the first direction, with a predetermined spacing from the first line in a second direction orthogonal to the first direction, and that the first protrusions in the first line and the second line are arranged with a shift half the pitch P in the first direction.

According to this application example, the first protrusions in the first line and the second line are arranged with a shift half the pitch P in the first direction. Therefore, an effective space to discharge moisture, for example, sweat, can be secured between the first protrusions next to each other. Also, the number of lines where the first protrusions are arranged can be increased. That is, the number of first protrusions arranged can be increased. This can achieve a good feeling to the touch.

### Application Example 14

In the portable electronic device strap according to the application example, it is preferable that the first protrusions are provided on the strap main body.

According to this application example, a part of the case fitting section close to the case can come into tight contact with the subject, whereas the strap main body having a large contact area can be prevented from coming into tight contact with the subject by the first protrusions provided thereon. This can achieve secure tight contact of the case and a good feeling to the touch of the strap main body.

### Application Example 15

It is preferable that the portable electronic device strap according to the application example has a second surface which faces opposite to the subject side when the case is fixed to the subject, that the second surface is provided with a plurality of second protrusions, and that the second protrusions have a pitch P2 and an outer dimension D2 that are different from those of the first protrusions.

According to this application example, the plurality of second protrusions provided on the second surface are provided with the pitch P2 and the outer dimension D2, which are different from those of the first protrusions. Thus, the adhesion of fluff and dirt due to contact with the external environment such as clothes, which tends to occur on the second surface situated on the surface side opposite to the subject side, can be made less likely to occur.

### Application Example 16

In the portable electronic device strap according to the application example, it is preferable that the first surface and the second surface are made up of different materials from each other.

According to this application example, for example, the first surface and the second surface can be made different from each other in softness or color. For example, if the second surface is made up of a harder material than the first surface, the strength of the second surface increases. This can increase the strength while maintaining a good feeling of wearing of the first surface. Meanwhile, for example, if the second surface is in a color that makes dirt less visible than on the first surface, a portable electronic device strap that has better design and achieves a good feeling of wearing can be provided.

### BRIEF DESCRIPTION OF THE DRAWINGS

The invention will be described with reference to the accompanying drawings, wherein like numbers reference like elements.

FIG. 1 is a top-side (surface-side) perspective view showing a configuration example according to an embodiment of a wrist device as a portable electronic device.

FIG. 2 is a bottom-side (back-side) perspective view) of the wrist device.

FIG. 3 is an outer view showing an example of wearing the wrist device.

FIG. 4 is an enlarged view of a Q part in FIG. 1, showing an arrangement example 1 of first protrusions provided on a strap for fitting the wrist device.

FIG. 5 is a cross-sectional view taken along A-A in FIG. 4, showing the arrangement example 1 of the first protrusions.

FIG. 6 is a list showing the correlation between evaluation test samples of the first protrusions and static frictional resistance (μs).

FIG. 7 is a graph showing the results of sensory evaluation of the evaluation test samples shown in FIG. 6.

FIG. 8 is a list showing the result of evaluation with respect to deterioration with time of the first protrusions according to the arrangement example 1 shown in FIG. 4.

FIG. 9 is an enlarge view of the Q part in FIG. 1, showing an arrangement example 2 of the first protrusions.

FIG. 10 is a cross-sectional view taken at the same position as FIG. 5, showing an arrangement example of second protrusions provided on a strap, as an application example 1 of the strap for fitting the wrist device.

FIG. 11 is a cross-sectional view taken at the same position as FIG. 5, showing an application example 2 of the strap for fitting the wrist device.

FIG. 12 is an enlarged view of the Q part in FIG. 1, showing a modification of the first protrusions.

FIG. 13 is a cross-sectional view taken along B-B in FIG. 12, showing the modification of the first protrusions.

### DESCRIPTION OF EXEMPLARY EMBODIMENTS

Hereinafter, an embodiment will be described. The embodiment described below should not unduly limit the content of the invention described in the appended claims. Not all the components described in the embodiment are essential components of the invention.

### Embodiment of Wrist Device

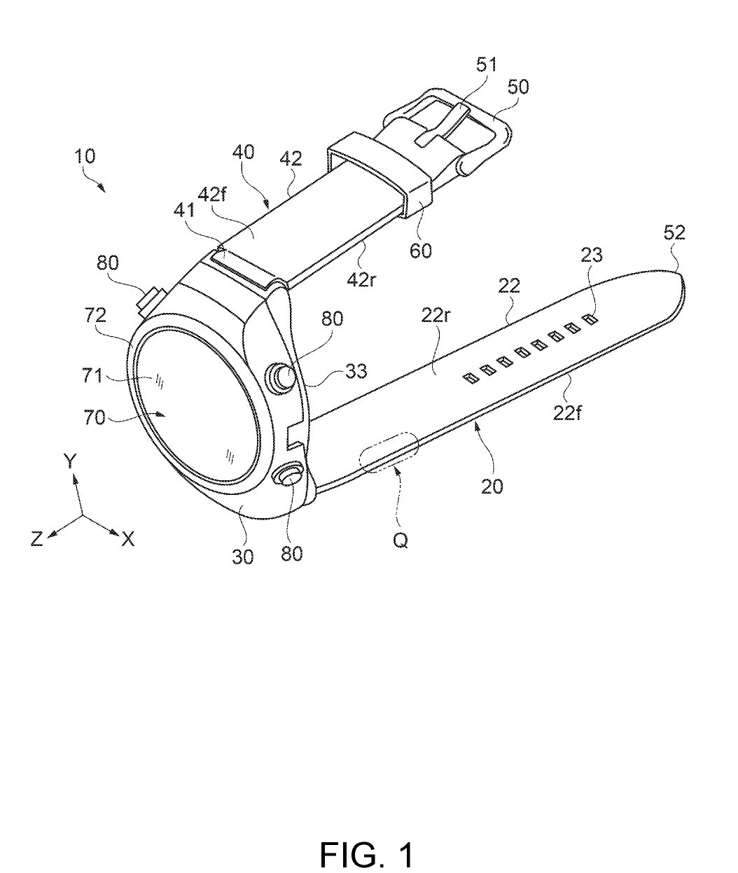

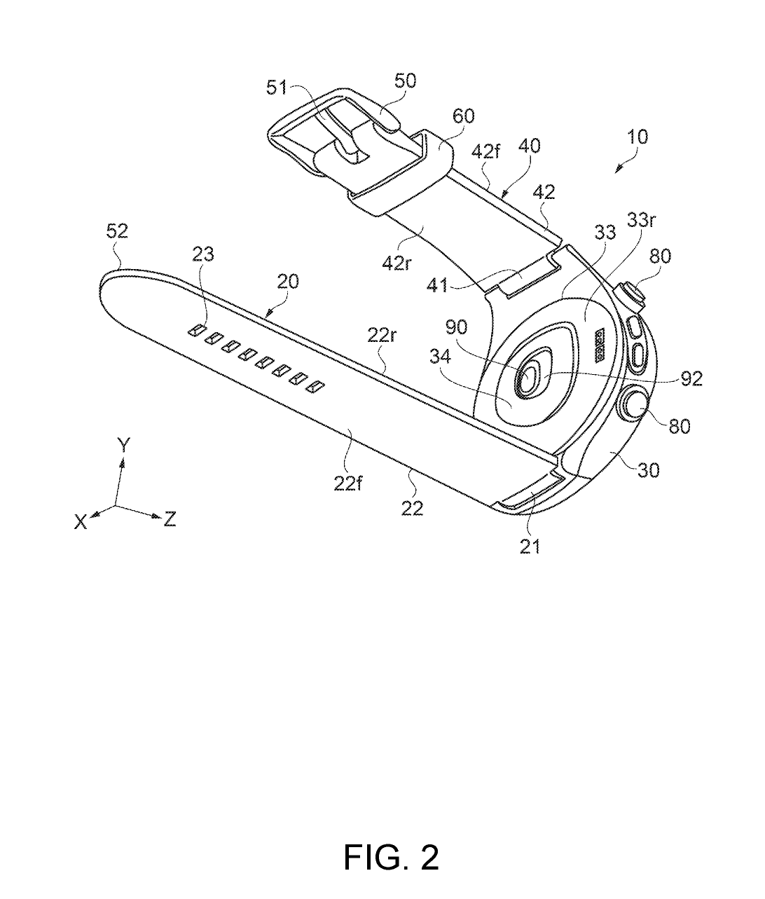

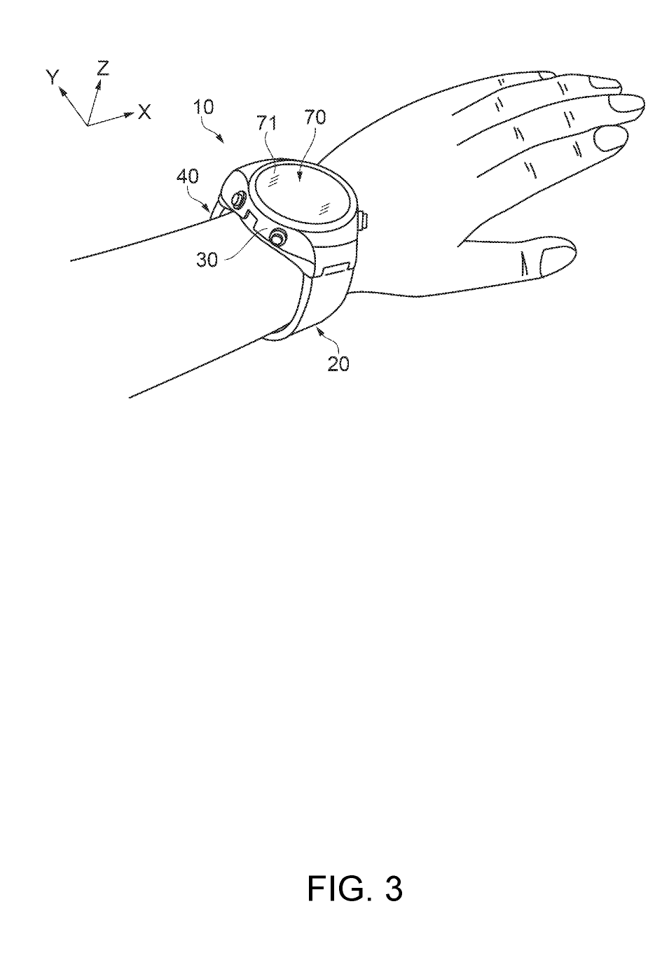

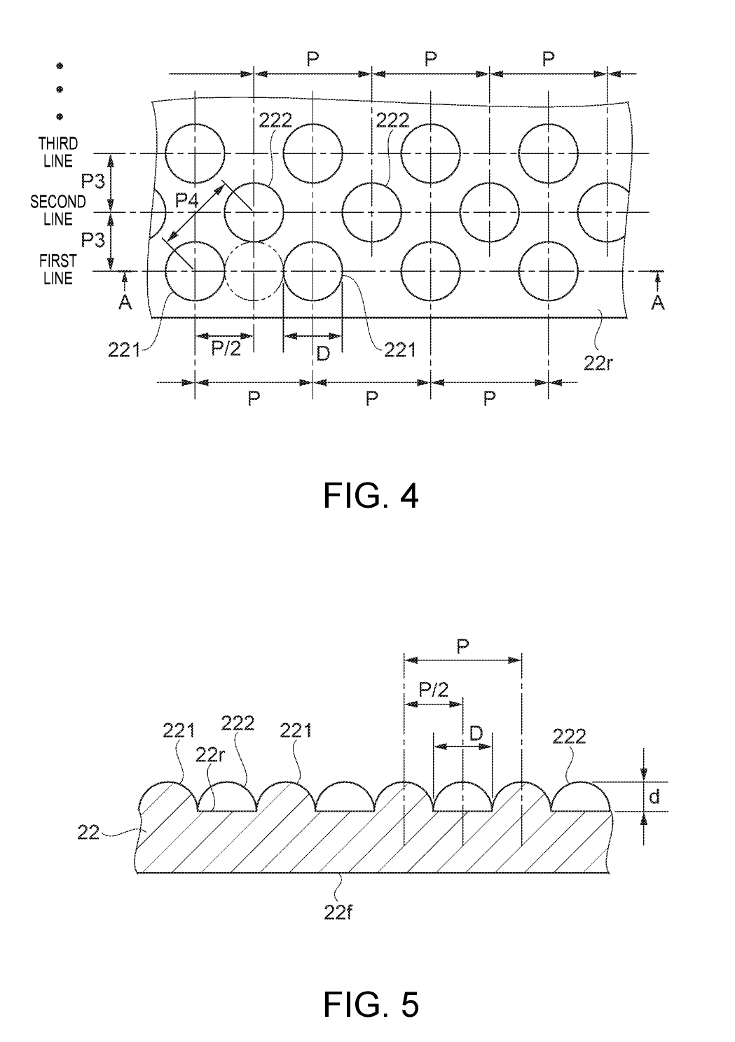

A configuration example of a wrist device as a portable electronic device according to an embodiment of the invention will be described, referring to FIGS. 1, 2, 3, 4, and 5. FIG. 1 is a top-side (surface-side) perspective view showing a configuration example according to the embodiment of the wrist device as a portable electronic device. FIG. 2 is a bottom-side (back-side) perspective view) of the wrist device. FIG. 3 is an outer view showing an example of wearing the wrist device. FIG. 4 is an enlarged view of a Q part in FIG. 1, showing an arrangement example 1 of first protrusions provided on a strap for fitting the wrist device. FIG. 5 is a cross-sectional view taken along A-A in FIG. 4, showing the arrangement example 1 of the first protrusions.

In this specification, the display surface side of the wrist device is defined as a surface side, and the side opposite to the display surface (side that comes into contact with the subject when the device is worn) is defined as a bottom side or a back side. In FIGS. 1, 2, and 3, three axes that are orthogonal to each other are described, using an X-axis, a Y-axis, and a Z-axis, for the sake of convenience. An axis along the direction in which a display unit 70 and an optical sensor unit 90, described later, are arranged next to each other, that is, an axis perpendicular to the display surface of the display unit 70, is defined as the Z-axis. The direction in which a pair of strap sections (first strap section 20 and second strap section 40) arranged to hold the case from both side are arranged next to each other is defined as the Y-axis. In the description below, the term “axial direction” means “a direction along an axis”.

As shown in FIGS. 1, 2, and 3, a wrist device 10 as a portable electronic device includes a second case 33 which is worn, for example, on the wrist of a subject (wearer) as shown in FIG. 3 and which is provided with an optical sensor unit 90 for detecting biological information of the subject, and a first case 30 situated on the side opposite to the subject side. On both sides along the Y-axis direction of a case including the second case 33 and the first case 30, a strap section (first strap section 20 and second strap section 40) is connected.

The configuration of the wrist device 10 will now be described in more detail. The wrist device 10 includes: the first strap section 20 and the second strap section 40 forming a portable electronic device strap, the case including the second case 33 and the first case 30, a fixing part (free loop, keeper) 60, a connecting part (buckle) 50 including a tongue 51, a display unit 70, an operation unit 80, and the optical sensor unit 90 as a detection unit. However, the wrist device 10 is not limited to the configuration shown in FIGS. 1, 2, and 3. Various modifications can be made such as omitting a part of the components or adding another component.

The second case 33 and the first case 30 forming the case can be made up of a metal such as stainless steel, or a resin such as polycarbonate (PC), polystyrene (PS), or ABS resin. The case need not be separable into the first case 30 and the second case 33. A case of a unified structure (equivalent to the first case 30), and a back lid (equivalent to the second case 33) on the side to be worn by the user of the case maybe provided. Also, a case equivalent to the first case 30 and a back lid equivalent to the second case 33 may be provided as an integrated structure.

A bezel 72 is provided on the top side (first case 30), which is the side opposite to the side worn on the wrist of the user as the subject of the wrist device 10. Also, a glass plate 71 is provided which is a light-transmissive member as a top plate part (outer wall) to be arranged on the inner side of the bezel 72 and protect the internal structure. The wrist device 10 has the display unit 70 including a display panel (not illustrated) provided directly below the glass plate 71. The user can view a display on the display unit 70 via the glass plate 71. That is, the wrist device 10 according to the embodiment may display detected biological information on the display unit 70 and present this display to the user from the top side of the wrist device 10.

While an example where the glass plate 71 forms the top plate part of the wrist device 10 is described here, the top plate part may be made up of materials other than glass, such as transparent plastics, provided that it is a transparent member through which the user can view the display unit 70 and which has enough strength to be able to protect the configuration included inside (the internal space of) the first case 30 and the second case 33 such as the display unit 70. Also, while a configuration example having the bezel 72 is described, a configuration without the bezel 72 may also be employed.

On a case outer surface of the second case 33 situated on the subject side, a protruding part 34 protruding from the case outer surface is provided. The optical sensor unit 90 as a detection unit for detecting biological information of the subject is exposed at a top part of the protruding part 34.

The optical sensor unit 90 is accommodated in a detection window part 92 provided in the protruding part 34 protruding in a convex form from a back surface 33r, which is the case outer surface of the second case 33 situated on the subject side. The optical sensor unit 90 accommodated in the detection window part 92 is housed in the case. In other words, the optical sensor unit 90 housed in the case partly protrudes to the subject side (in the Z-axis direction) from the back surface 33r. The detection window part 92 is a transparent convex lens-shaped member. In a preferred example, the detection window part 92 uses a transparent resin. The detection window part 92 is formed at a top part extending from the back surface 33r via a sloped surface and has the optical sensor unit 90 accommodated at a center part thereof. The optical sensor unit 90 detects biological information of the subject, in the state of being pressed against the subject with the detection window part 92.

The optical sensor unit 90 is provided with a light emitting unit and a light receiving unit, though not illustrated. The light emitting unit emits light to the subject. The light receiving unit receives light (reflected light) from the subject . For example, when the light emitting unit emits light and the light is reflected by the subject (blood vessel or the like), the light receiving unit can receive the reflected light and thus can detect biological information. The light receiving unit can be implemented by a light receiving element such as a photodiode. The light emitting unit can be implemented by a light emitting element such as an LED.

A pulse monitor will now be taken as an example. The light from the light emitting unit travels inside the subject and is then diffused or dispersed by the epidermis, the dermis, and the subcutaneous tissues or the like. Subsequently, this light reaches a blood vessel (detection target site) and is reflected there. In this case, a part of the light is absorbed by the blood vessel. Due to the influence of pulses, the absorptance of the blood vessel to light changes and the amount of light of the reflected light changes as well. The light receiving unit receives this reflected light and detects a change in the amount of light. Thus, pulse rates or the like as biological information can be detected.

The operation unit 80 including a plurality of buttons that enable, for example, switching between detection modes of the optical sensor unit 90 and switching between display modes displayed on the display unit 70, is provided on the lateral side of the wrist device 10. The display unit 70 displays various kinds of information such as biological information detected by the optical sensor unit 90 and motion information detected by a body motion sensor unit, described later, accommodated inside the case. The operation unit 80 accepts an operation by the user.

The display unit 70 including a display panel (not illustrated) such as a liquid crystal panel, a direction sensor (not illustrated) and an acceleration sensor (not illustrated) included in the body motion sensor unit as a detection unit, and a battery (not illustrated) as a power supply unit, and the like, are accommodated in the internal space formed by the first case 30, the second case 33, and the glass plate 71 or the like. Also, in this internal space, a control unit (not illustrated) which processes data detected by the optical sensor unit 90 and the body motion sensor and controls the content of display on the display unit 70, and a circuit board (not illustrated) on which these components are loaded, and the like, are accommodated. However, the wrist device 10 is not limited to the above configuration. For example, a location information acquisition unit using the GPS (Global Positioning System) as an example of a positioning system using location information satellites, called the GNSS (Global Navigation Satellite System), and other sensors or vibrators such as a barometric pressure sensor to calculate height above sea level or a temperature sensor to measure temperature may be added.

The first strap section 20 includes a case fitting section 21, a strap main body 22 extending from the case fitting section 21 to a tip 52, which is an end part, and adjustment holes 23 arrayed on the tip 52 side of the strap main body 22. The first strap section 20 is attached at the case fitting section 21 to one end of the first case 30. The strap main body 22 has a first surface 22r that faces the subject when the case is fixed to the subject, and a second surface 22f that faces opposite to the first surface 22r. On the first surface 22r, a plurality of first protrusions 221, 222 (see FIGS. 4 and 5), described later, are arranged as so-called “embossing”. Also, a plurality of seconds protrusions 270 (see FIG. 10), described later, can be arranged on the second surface 22f as so-called “embossing”.

The second strap section 40 includes a case fitting section 41, a strap main body 42 extending from the case fitting section 41 to the connecting part (including the connecting part (buckle) 50 and the tongue 51), and the fixing part (free loop, keeper) 60 attached to the connecting part side of the strap main body 42. The second strap section 40 is attached at the case fitting section 41 to the other end of the first case 30. The strap main body 42 has a first surface 42r that faces the subject when the case is fixed to the subject, and a second surface 42f that faces opposite to the first surface 42r. On the first surface 42r, a plurality of first protrusions 221, 222 (see FIGS. 4 and 5), described later, are arranged. Also, a plurality of seconds protrusions 270 (see FIG. 10), described later, can be arranged on the second surface 42f.

The wrist device 10 has the first strap section 20 and the second strap section 40 connected to each other, using the tip 52 and the adjustment holes 23 of the first strap section 20 and the connecting part (including the connecting part (buckle) 50 and the tongue 51) of the second strap section 40, and is thus worn with the second case 33 in tight contact with the wrist of the user (subject). More specifically, to fit the wrist device 10 to the wrist of the user (subject) with the first strap section 20 and the second strap section 40, the end part (tip 52) of the first strap section 20 is inserted into the connecting part (buckle) 50 provided to be rotatable via a shaft pin (not illustrated) of the second strap section 40. Then, at a position where predetermined contact strength is secured, the tongue 51 is inserted through one of the adjustment holes 23 via a part of the connecting part (buckle) 50 fitted to be rotatable about the shaft pin (not illustrated).

Thus, the wrist device 10 is worn, tightened as needed to bring the optical sensor unit 90 into tight contact with the wrist (subject) with proper strength. That is, with the positional setting between the tongue 51 and the adjustment hole 23 and a proper pressing force based on the elasticity of the first strap section 20 and the second strap section 40, the optical sensor unit 90 can be brought into tight contact with the wrist (subject) with proper strength.

The wrist device 10 is an electronic device which is worn by the user (wearer) not only when doing exercises such as walking or running but constantly and thus aims to measure biological information such as the pulse rate of the wearer in everyday life. Therefore, the wrist device 10 needs to be able to measure biological information in a stable state regardless of the attitude and movement of the wearer and with little burden (for example, an uncomfortable feeling due to the weight, size, shape, tightening force, and tight contact of the device) on the user (wearer) even if the user wears the device for a long period.

Therefore, it is desirable that the first strap section 20 and the second strap section 40 are properly flexible and elastic, highly durable, and gentle on the skin (less irritating to the skin) in order to perform tightening as needed to bring the optical sensor unit 90 into tight contact with the wrist (subject) with proper strength. As a material forming the first strap section 20 and the second strap section 40 having such characteristics, for example, a silicone rubber (silicone resin) or elastomer material can be used suitably.

For the first strap section 20 and the second strap section 40 made of a silicon rubber or elastomer material, a plurality of first protrusions 221, 222 as shown in FIGS. 4 and 5 are provided on the first surfaces 22r, 42r facing the subjects side of the strap main bodies 22, 42 in order to cope with so-called high tackiness, that is, a characteristic that the surface is gummy and sticky. While FIGS. 4 and 5 show an enlarged view of a Q part in FIG. 1, which is a part of the first strap section 20, similar first protrusions 221, 222 are provided on the second strap section 40 as well.

It is preferable that the plurality of first protrusions 221, 222 are provided on the first surfaces 22r, 42r facing the subject side of the strap main bodies 22, 42. As the plurality of first protrusions 221, 222 are thus provided on the first surfaces 22r, 42r facing the subject side of the strap main bodies 22, 42, parts of the case fitting sections 21, 41 close to the case can come into tight contact with the subject and the strap main bodies 22, 42 having a large contact area can be prevented from coming into tight contact with the subject by the first protrusions 221, 222 provided there. This can achieve secure tight contact of the case and a good feeling of wearing (good feeling to the touch) of the strap (strap main bodies 22, 42).

Now, an arrangement example 1 of the first protrusions 221, 222 provided on the strap will be described, referring to FIGS. 4 and 5. As shown in FIGS. 4 and 5, a plurality of first protrusions 221, 222 are provided on the first surface 22r of the strap main body 22. Each of the first protrusions 221, 222 protrudes hemispherically with a height d from the first surface 22r and has a circular shape with an outer dimension D (diameter) as viewed in a plan view seen from an axial direction perpendicular to the first surface 22r. The height d of the first protrusions 221, 222 can also be considered as the depth d from the top part of the first protrusions 221, 222 to the first surface 22r.

The first protrusions 221 forming a first line are arranged with a pitch P in a first direction, that is, in this embodiment, in a direction along the direction in which the strap main body 22 extends. The pitch P in this case is the distance between the centers of the first protrusions 221 situated next to each other, where the centroid of each first protrusion 221 is defined as the center, as viewed in a plan view of the first protrusions 221 from an axial direction perpendicular to the first surface 22r.

The first protrusions 222 forming a second line are arranged with a pitch P3 which is a predetermined spacing from the first line (in this embodiment, the same distance as the diameter D of each first protrusion 221, which is shorter than the pitch P (P3=0.15 mm) is set) in a second direction orthogonal to the first direction, that is, in this embodiment, in a direction along the direction of width of the strap main body 22, and with a pitch P in the first direction. As with the first protrusions 221, the pitch P in this case is the distance between the centers of the first protrusions 222 situated next to each other, where the centroid of each first protrusion 222 is defined as the center, as viewed in a plan view of the first protrusions 222 from an axial direction perpendicular to the first surface 22r.

In the second line, the first protrusions 222 forming the second line are arranged with a shift half the pitch P in the first direction from the first protrusions 221 forming the first line. Since the first protrusions 221 in the first line and the first protrusions 222 in the second line are thus arranged with a shift half the pitch P from each other in the first direction, an effective space to discharge moisture such as sweat can be secured between the first protrusions 221, 222 situated next to each other.

As the spacing between the second line and the first line is set to be shorter than the pitch P, the number of line in which the first protrusions 221, 222 are arranged can be increased. That is, the number of first protrusions 221, 222 arranged can be increased. This can achieve a good feeling to the touch.

Here, the outer dimension D of each of the first protrusions 221, 222 is an outer dimension of each of the first protrusions 221, 222 in a direction of connecting the centers of two or more first protrusions 221 or two or more first protrusions 222. It is preferable that the outer dimension D is 0.03 mm≤D≤1.0 mm. In this embodiment, the outer dimension D of each of the first protrusions 221, 222 is set to 0.15 mm. The lower limit of 0.03 mm is a minimum value that enables the formation of the first protrusions 221, 222. The upper limit of 1.0 mm is set as a limit value below which a poor feeling of wearing such as stickiness due to the increase in the contact area with the skin does not occur.

It is also preferable that the pitch P is 0.005 mm≤P≤0.3 mm. In this embodiment, the pitch P is set to 0.3 mm. This is because, with the first protrusions 221, 222 formed with a smaller pitch than a fingerprint (palm print), whose pitch is considered to be 0.3 mm to 0.5 mm, the fingerprint (palm print) and the first protrusions 221, 222 are less likely to be fitted with each other when the first strap section 20 and the second strap section 40 are placed on the wrist. In other words, not all the first protrusions 221, 222 enter grooves of the fingerprint (palm print), constantly leaving a gap between the surface of the wrist and the first surfaces 22r, 42r of the strap main bodies 22, 42. This makes the first surfaces 22r, 42r of the strap main bodies 22, 42 less likely to come into direct contact with the skin and thus can maintain a good feeling to the touch.

It is also preferable that the depth d from the top part of the first protrusions 221, 222 to the first surface 22r (in other words, the height d from the first surface 22r) satisfies 0.005 mm≤d≤0.2 mm. Setting the depth d from the top part of the first protrusions 221, 222 to the first surface 22r within such a range can secure enough strength of the first protrusions 221, 222 not to fall off even if an external force is applied to the first protrusions 221, 222. This can improve the durability of the strap sections. In this embodiment, the depth d (height d) is set to d=0.05 mm.

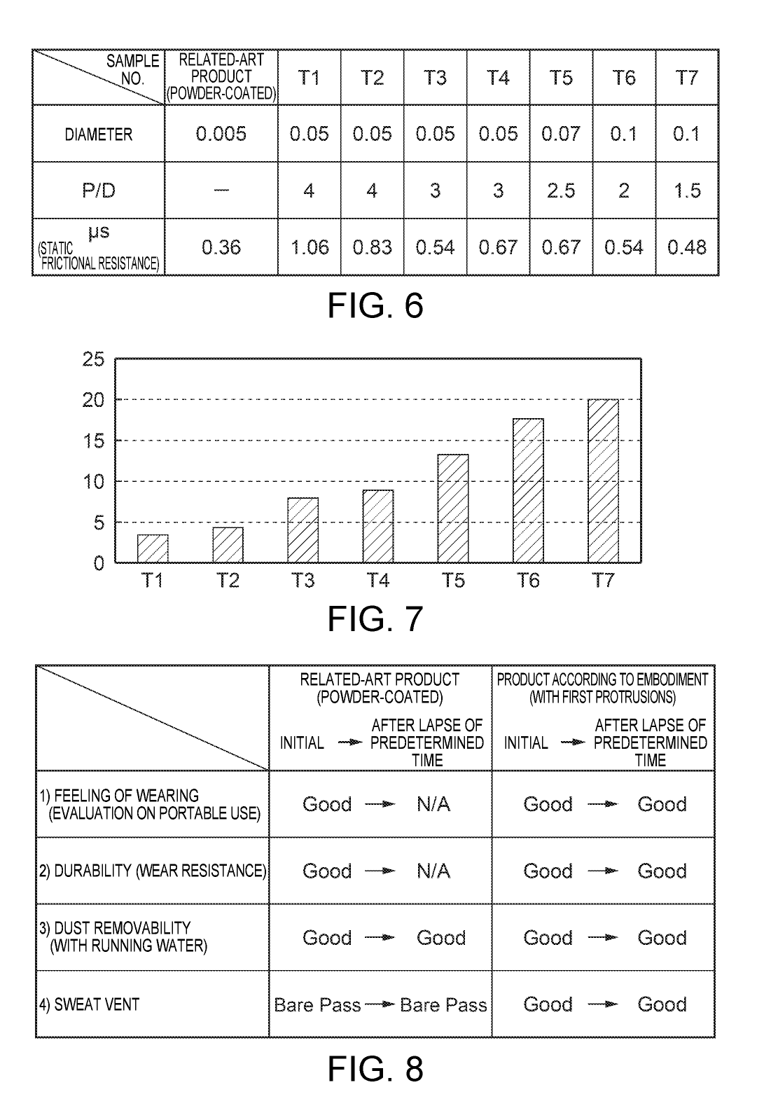

Also, with respect to the arrangement of the first protrusions 221, 222, the inventors have found that setting the outer dimension D of the first protrusions 221, 222 and the ratio P/D of the outer dimension D and the arrangement pitch P within a predetermined range can improve the durability of the first protrusions 221, 222 and can also improve the feeling of wearing on the wrist, which is a wearing part of the wearer (living body). A preferable range of arrangement of the first protrusions 221, 222 will now be described, referring to FIGS. 6, 7, and 8. FIG. 6 is a list showing the correlation between evaluation test samples of the first protrusions and static frictional resistance (μs). FIG. 6 shows the correlation between test samples T1 to T7, combined with the outer dimension D and the ratio P/D of the outer dimension D and the pitch P, and static frictional resistance (μs). FIG. 7 is a graph showing the results of sensory evaluation of the evaluation test samples T1 to T7 shown in FIG. 6. FIG. 8 is a list showing the result of evaluation with respect to deterioration with time of the first protrusions according to the arrangement example 1 shown in FIG. 4.

The test samples T1 to T7 shown in the list of FIG. 6 are samples each made up of a silicone resin material whose surface is provided with the first protrusions 221, 222 with a predetermined outer dimension D and pitch P. The list shown in FIG. 6 also describes a powder-coated product according to the related art as a comparative example. The powder-coated product according to the related art is made up of a silicone resin material coated with a powder 0.005 mm in diameter and has a static frictional resistance (μs) of 0.36 in an initial (unused) state with high smoothness. Here, the letter μ (mu) is the coefficient of static friction, and the letter s is the area. A static frictional resistance (μs) of 0.8 or higher is considered to cause reduced smoothness, increased stickiness, and a poor feeling of wearing. The test samples T1 and T2 with a P/D of 4 are shown to have a static frictional resistance (μs) of 0.8 or higher. In contrast, the test samples T3 to T7 with a P/D of 3 or lower are shown to have a static frictional resistance (μs) lower than 0.8.

Specifically, the test sample T3 with a P/D of 3 is shown to have a static frictional resistance (μs) of 0.54. The test sample T4, again with a P/D of 3, is shown to have a static frictional resistance (μs) of 0.67. The test sample T5 with a P/D of 2.5 is shown to have a static frictional resistance (μs) of 0.67. The test sample T6 with a P/D of 2 is shown to have a static frictional resistance (μs) of 0.54. The test sample T7 with a P/D of 1.5 is shown to have a static frictional resistance (μs) of 0.48. In this way, if the P/D is 3 or lower, a static frictional resistance (μs) of 0.8 or lower, which is the range of high smoothness, can be achieved.

Sensory evaluation has been carried out in which several hundred subjects wear the wrist device 10 with the strap sections (first strap section 20 and second strap section 40) having the first protrusions 221, 222 similar to those of the test samples T1 to T7 and vote for a sample number which each subject feels the most comfortable to wear. Then, the relation between the above static frictional resistance (μs) and the sensory evaluation by the subjects has been examined. The graph of FIG. 7 shows the results of the examination. The graph shown in FIG. 7 is an indicator-like bar chart showing the number of votes for each of the test samples T1 to T7 on the horizontal axis. In this graph, a longer bar indicates that a larger number of subjects determine that the test sample has a good feeling of wearing.

As a result of the sensory evaluation by the subjects, the number of subjects who feel the test sample the most comfortable to wear clearly increases from the test samples T1, T2 with a P/D=4 toward the test sample T7 with a P/D of 1.5, as shown in the graph of FIG. 7. From this graph, it can be confirmed that there is a correlation between the smoothness and the feeling of wearing of the first strap section 20 and the second strap section 40, that is, between the value of P/D, which is the ratio of the outer dimension D and the pitch P of the first protrusions 221, 222 provided on the first strap section 20 and the second strap section 40 considered to correlate with smoothness, and the sensory evaluation by the subjects, as described with reference to FIG. 6, and that the sense of wearing by the subjects is improved as the P/D value becomes smaller.

As a result of an extensive study including the results of evaluation described with reference to FIGS. 6 and 7, the inventors have found that it is preferable to set the outer dimension of the first protrusions 221, 222 provided on the first strap section 20 and the second strap section 40 to a range of 0.03 mm≤D≤1.0 mm as shown in the arrangement example 1 and to arrange the first protrusions 221, 222 in such a way as that the ratio P/D of the pitch P with which two first protrusions 221, 222 are arranged and the outer dimension D of the first protrusions 221, 222 satisfies 1<P/D≤3. Based on this finding, in this embodiment, the outer dimension D of the first protrusions 221, 222 on the first strap section 20 and the second strap section 40 is set to D=0.15 mm and the pitch P is set to P=0.3 mm, resulting in P/D=0.3/0.15=2.

In this way, the first protrusions 221, 222, arranged on the first strap section 20 and the second strap section 40 with the outer dimension D of the first protrusions 221, 222 and the ratio P/D of the pitch P and the outer dimension D satisfying 0.03 mm≤D≤1.0 mm and 1<P/D≤3, can secure a proper spacing between the subject (skin of the wrist) and the strap main bodies 22, 42 when the case is fixed to the subject. Also, the provision of such first protrusions 221, 222 can secure proper smoothness between the strap main bodies 22, 42 and the subject and thus can improve the feeling of wearing. Moreover, the outer dimension D satisfying 0.03 mm≤D≤1.0 mm can secure a proper size and strength of the first protrusions 221, 222, thus making the first protrusions 221, 222 less likely to be separated due to abrasion or friction. Thus, even if the device is used continuously for measurement over a long period with the optical sensor unit 90 as a detection unit in tight contact with the subject, comfortable wearing can continue with reduced burden on the wearer.

Next, the results of evaluation on the durability (deterioration with time) of the portable electronic device strap (first strap section 20 and second strap section 40) according to this embodiment will be described, referring to FIG. 8. The list shown in FIG. 8 shows the results of evaluating the state of deterioration, in terms of the four items given below, of a strap section made up of a silicone resin material coated with a powder 0.005 mm in diameter, which is a related-art powder-coated product as a comparative example, and the portable electronic device strap (first strap section 20 and second strap section 40) provided with the first protrusions 221, 222 with the outer dimension D set to 0.15 mm and the pitch P set to 0.3 mm (P/D=2), after use under the same conditions for a predetermined time. The four items are: 1. feeling of wearing based on evaluation on portable use; 2. durability (evaluation on wear resistance); 3. dust removability (with running water); and 4. sweat vent.

As shown in the list of FIG. 8, the confirmation results show that the related-art product as a comparative example deteriorates with time in terms of the items of 1. feeling of wearing and 2. durability, which are in good condition (Good) in the initial state but turn N/A (Not Acceptable), as described above. In contrast, the product according to the embodiment maintains a good condition (Good) in terms of the evaluation items 1 to 4 even after the lapse of the predetermined time. In this way, it has been confirmed that the strap sections (first strap section 20 and second strap section 40) provided with the first protrusions 221, 222 in this embodiment are durable in terms of each of the evaluation items.

Also, by arranging the first protrusions 221, 222 on the strap sections (first strap section 20 and second strap section 40) in such a way that the outer dimension of the first protrusions 221, 222 satisfies 0.03≤mm D≤0.8 mm and that the ratio P/D of the pitch P with which two first protrusions 221, 222 are arranged and the outer dimension D of the first protrusions 221, 222 satisfies 1.4≤P/D≤3, it is possible to further reduce the burden on the wearer and thus achieve more comfortable wearing. The ratio P/D=1.4 in this case corresponds to the pitch P4=0.15×√2, which is the distance between the first protrusions 221 in the first line and the first protrusions 222 in the second line, the outer dimension D=0.15 of the first protrusions 221, 222, thus resulting in the ratio P/D=0.141, in the arrangement example of the first protrusions 221, 222 in this embodiment, as shown in FIG. 4. The configuration of the strap having this P/D value has good results in terms of the deterioration with time, as shown in FIG. 8.

Other members forming the first strap section 20 and the second strap section 40 can be, for example, natural rubber, carbon black-mixed rubber, isoprene rubber, butadiene rubber, styrene-butadiene rubber, chloroprene rubber, nitrile rubber, polyisobutylene, ethylene-propylene rubber, chlorosulfonated polyethylene rubber, acrylic rubber, fluorine rubber, epichlorohydrin rubber, urethane rubber (urethane resin), polyurethane rubber (polyurethane resin), styrene-based elastomer, olefin-based elastomer, polyvinyl chloride-based elastomer, polyester-based elastomer, polyurethane-based elastomer, silicone-based elastomer, amide-based elastomer, Nylon-based elastomer, dynamically vulcanized elastomer or the like, or a mixture of these. Other materials that can be used include synthetic leather, real skin, real leather, polyethylene made up of thermoplastic resin, polypropyrene, polyolefin such as ethylene-vinyl acetate copolymer, modified polyolefin, polyamide (for example, Nylon 6, Nylon 46, Nylon 66, Nylon 610, Nylon 612, Nylon 11, Nylon 12, Nylon 6-12, Nylon 6-66), thermoplastic polyimide, liquid crystal polymer such as aromatic polyester, polyphenylene oxide, polyphenylene sulfide, polycarbonate, polymethylmethacrylate, polyether, polyether ketone, polyether imide, polyacetal, various thermoplastic elastomers such as styrene-based, polyolefin-based, polyvinyl chloride-based, polyurethane-based, polyester-based, polyamide-based, polybutadiene-based, trans polyisoprene-based, fluorine rubber-based, and chlorinated polyethylene-based thermoplastic elastomer, and copolymer, polymer blend, polypermalloy or the like made up mainly of these. One of these materials can be used. Also, two or more can be mixed together.

### Another Arrangement of First Protrusions

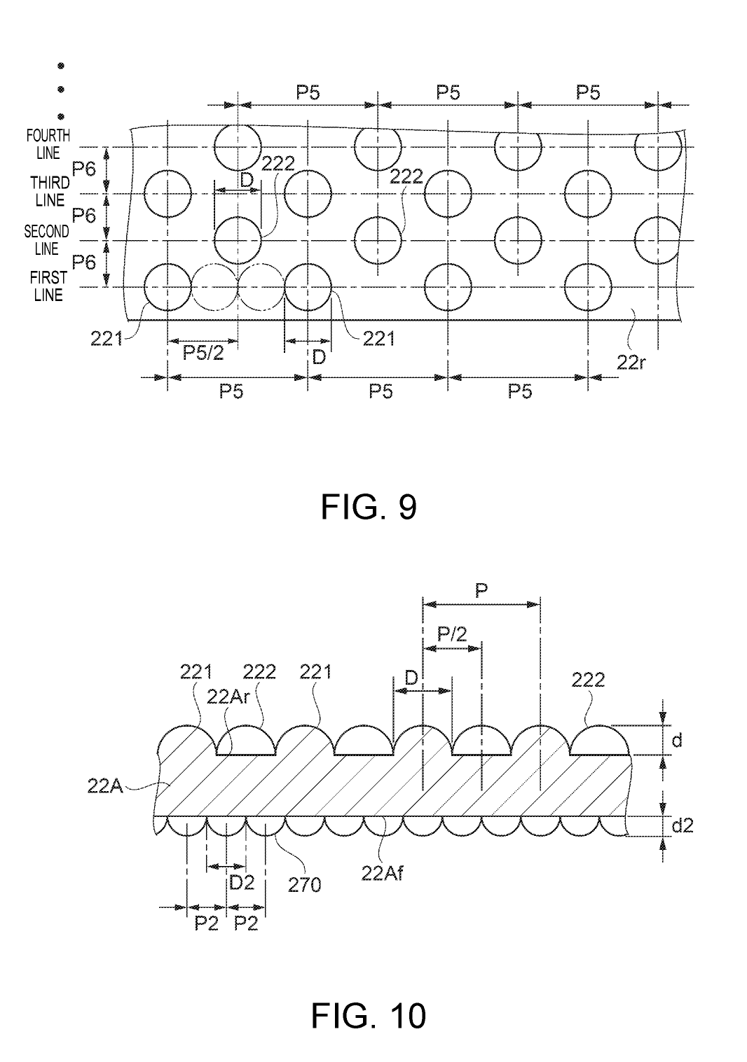

Now, another arrangement example of the first protrusions 221, 222 will be described, referring to FIG. 9. An arrangement example 2 of the first protrusions 221, 222 has a different pitch P5 from that of the arrangement example 1 described with reference to FIGS. 4 and 5. Such an arrangement of the first protrusions 221, 222 has effects similar to those of the arrangement example 1 described with reference to FIGS. 4 and 5. FIG. 9 is an enlarged view of the Q part in FIG. 1, showing the arrangement example 2 of the first protrusions.

As shown in FIG. 9, in the arrangement example 2 of the first protrusions 221, 222, a plurality of first protrusions 221, 222 are provided on the first surface 22r of the strap main body 22, as in the foregoing arrangement example. Each of the first protrusions 221, 222 protrudes hemispherically with a height d (not illustrated) from the first surface 22r and has a circular shape with an outer dimension D (diameter) as viewed in a plan view seen from an axial direction perpendicular to the first surface 22r. The outer dimension D of the first protrusions 221, 222 in the arrangement example 2 is set to 0.1 mm. The height d (depth d) is set to 0.05 mm.

The first protrusions 221 forming a first line are arranged with a pitch P5 (in this arrangement example, P5=0.3 mm is set) in a first direction, that is, in this embodiment, in a direction along the direction in which the strap main body 22 extends. The pitch P5 in this case is the distance between the centers of the first protrusions 221 situated next to each other, where the centroid of each first protrusion 221 is defined as the center, as viewed in a plan view of the first protrusions 221 from an axial direction perpendicular to the first surface 22r.

The first protrusions 222 forming a second line are arranged with a pitch P6 which is a predetermined spacing from the first line (in this arrangement example, P6=0.1 mm is set) in a second direction orthogonal to the first direction, that is, in this embodiment, in a direction along the direction of width of the strap main body 22, and with a pitch P5 in the first direction. In the second line, the first protrusions 222 forming the second line are arranged with a shift half the pitch P5, that is, 0.15 mm in the first direction from the first protrusions 221 forming the first line.

In such an arrangement of the first protrusions 221, 222, the outer dimension D of the first protrusions 221, 222 is D=0.1 mm and the pitch P5 is P5=0.3 mm. Thus, the P/D value is P/D=3, satisfying 1<P/D≤3. Also, the pitch between the first protrusions 221 in the first line and the first protrusions 222 in the second line is 0.18 mm and the P/D value is P/D=1.8, satisfying 1<P/D≤3.

In the portable electronic device strap (first strap section 20 and second strap section 40) according to the above embodiment and the wrist device 10 using this strap, the first protrusions 221, 222 are arranged with the outer dimension D of the first protrusions 221, 222 and the ratio P/D of the pitch P and the outer dimension D satisfying 0.03 mm≤D≤1.0 mm and 1<P/D≤3. Therefore, a proper spacing can be secured between the subject (skin of the wrist) and the strap main bodies 22, 42 when the case is fixed to the subject. Also, the provision of such first protrusions 221, 222 can secure proper smoothness between the first protrusions 221, 222 (strap main body 22) and the subject and thus can improve the feeling of wearing. Moreover, the outer dimension D satisfying 0.03 mm≤D≤1.0 mm can secure a proper size and strength of the first protrusions 221, 222, thus making the first protrusions 221, 222 less likely to be separated due to abrasion or friction. Thus, even if the device is used continuously for measurement over a long period with the optical sensor unit 90 as a detection unit in tight contact with the subject, comfortable wearing can continue with reduced burden on the wearer.

### Application Examples of Portable Electronic Device Strap

Next, an application example 1 of the portable electronic device strap will be described, referring to FIG. 10. FIG. 10 shows an arrangement example of second protrusions provided on the strap, as the application example 1 of the strap for fitting the wrist device 10. FIG. 10 is a cross-sectional view taken at the same position as FIG. 5. While FIG. 10 shows the first strap section 20 (see FIG. 1) as a representative example, the second strap section 40 (see FIG. 1) can be configured similarly.

In the portable electronic device strap according to the application example 1, a plurality of first protrusions 221, 222 are provided on a first surface 22Ar of a strap main body 22A, as shown in FIG. 10. Each of the first protrusions 221, 222 protrudes hemispherically with a height d from the first surface 22Ar and has a circular shape with an outer dimension D (diameter) as viewed in a plan view seen from an axial direction perpendicular to the first surface 22Ar. The outer dimension D of the first protrusions 221, 222 is set to 0.15 mm. The height d (depth d) is set to 0.05 mm. The arrangement pattern including the pitch P of the first protrusions 221, 222 and the like is similar to the arrangement example 1 in the foregoing embodiment and therefore will not be described further in detail.

In the portable electronic device strap according to the application example 1, a plurality of second protrusions 270 are provided on a second surface 22Af of the strap main body 22A. The second surface 22Af is the surface that faces opposite to the subject side when the case of the wrist device 10 is fixed to the subject. The second surface 22Af is the surface opposite to the first surface 22Ar. The second protrusions 270 have a pitch P2 and an outer dimension D2 that are different from those of the first protrusions 221, 222. Specifically, each of the second protrusions 270 protrudes hemispherically with a height d2 (set to be lower than the first protrusions 221, 222) from the second surface 22Af and has a circular shape with an outer dimension D2 (smaller diameter than that of the first protrusions 221, 222) as viewed in a plan view seen from an axial direction perpendicular to the second surface 22Af. The second protrusions 270 are also arranged in the form of a lattice with a pitch P2, as viewed in a plan view seen from an axial direction perpendicular to the second surface 22Af.

In the portable electronic device strap according to the application example 1, the plurality of second protrusions 270 provided on the second surface 22Af are provided with the pitch P2 and the outer dimension D2, which are different from those of the first protrusions 221, 222. Particularly, if the outer dimension D2 and the pitch P2 are set to be smaller than those of the first protrusions 221, 222, the adhesion of fluff and dirt due to abrasion with clothes or contact with the external environment or the like, which tends to occur on the second surface 22Af situated on the surface side opposite to the subject side, can be made less likely to occur.

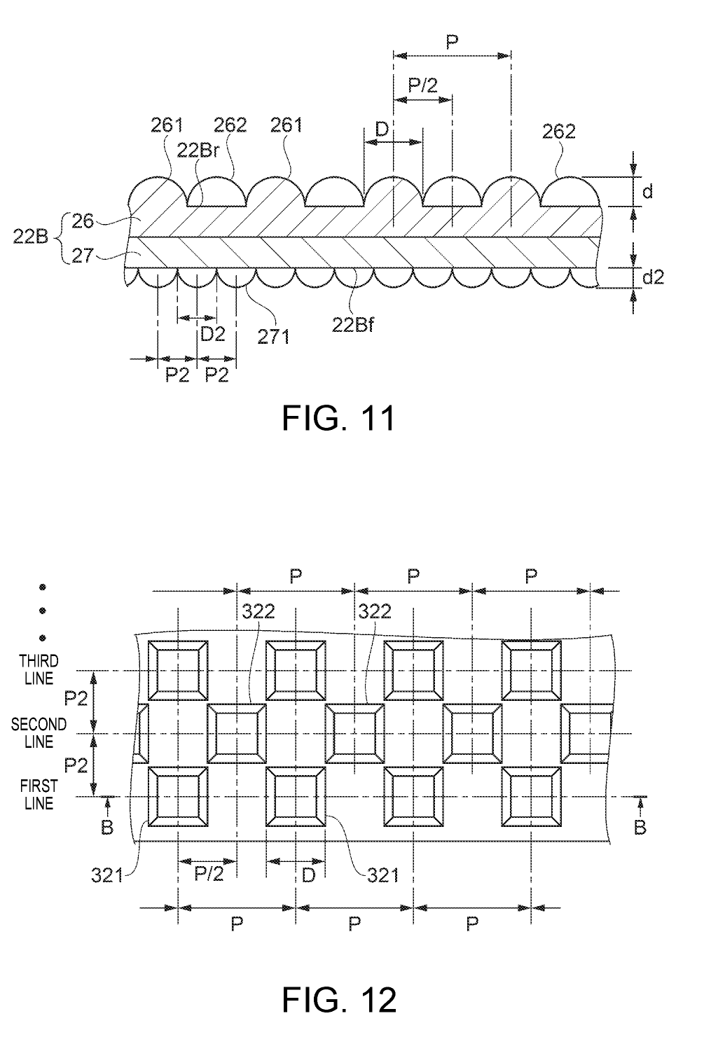

Next, an application example 2 of the portable electronic device strap will be described, referring to FIG. 11. FIG. 11 shows the arrangement example 2 of the strap for fitting the wrist device 10. FIG. 11 is a cross-sectional view taken at the same position as FIG. 5. While FIG. 11 shows the first strap section 20 (see FIG. 1) as a representative example, the second strap section 40 (see FIG. 1) can be configured similarly.

A strap main body 22B of the portable electronic device strap according to the application example 2 includes a first member 26 and a second member 27 joined to the first member 26 and made up of a different material from the first member 26, as shown in FIG. 11. The first member 26 has a first surface 22Br which faces the subject when the case of the wrist device 10 is fitted to the subject. The second member 27 has a second surface 22Bf which is the surface opposite to the subject side and thus opposite to the first surface 22Br when the case of the wrist device 10 is fixed to the subject.

Of the first surface 22Br and the second surface 22Bf, made up of different materials from each other, the first surface 22Br is provided with first protrusions 261, 262 similar to the first protrusions 221, 222 described in the application example 1, and the second surface 22Bf is provided with second protrusions 271 similar to the second protrusions 270 described in the application example 1. The arrangement pattern including the pitch P of the first protrusions 261, 262, and the second protrusions 271 are similar to those in the application example 1 and therefore will not be described further in detail.

In the portable electronic device strap according to the application example 2, for example, the first surface 22Br and the second surface 22Bf can differ from each other in softness or color. For example, if the second surface 22Bf is made up of a harder material than the first surface 22Br, the strength of the second surface 22Bf increases. This can increase the strength of the strap section while maintaining a good feeling of wearing of the first surface 22Br. Also, for example, if the second surface 22Bf is in a color that makes dirt less visible than on the first surface 22Br, the wrist device 10 that has better design and achieves a good feeling of wearing can be provided.

### Modifications of First Protrusions

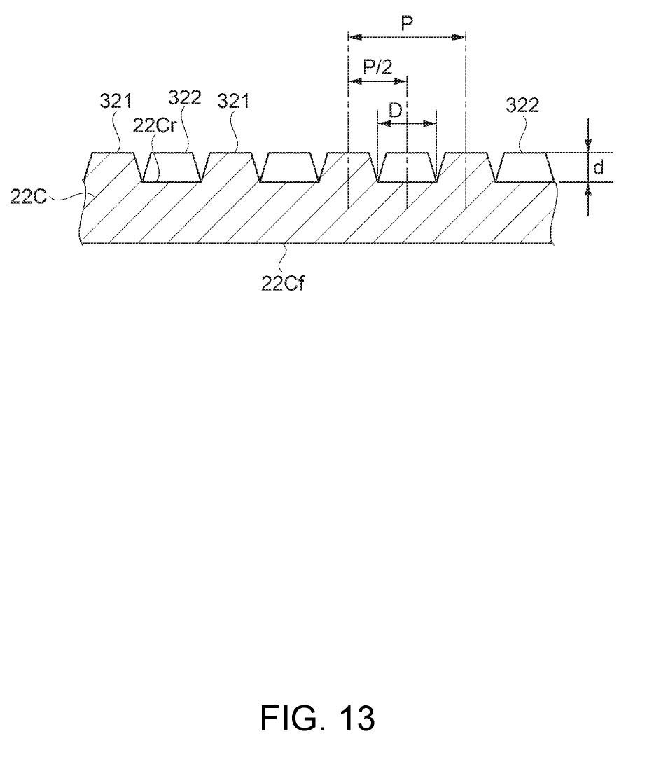

Next, modifications of the first protrusions 221, 222, 261, 262 provided on the portable electronic device strap will be described, referring to FIGS. 12 and 13. FIG. 12 is an enlarged view of the Q part in FIG. 1, showing a modification of the first protrusions. FIG. 13 is a cross-sectional view taken along B-B in FIG. 12, showing the modification of the first protrusions. While FIGS. 12 and 13 show a strap main body 22C equivalent to the strap main body 22 (see FIG. 1) of the first strap section 20 (see FIG. 1) as a representative example, the second strap section 40 (see FIG. 1) can be configured similarly.

The portable electronic device strap according to a modification 1 has the strap main body 22C, as shown in FIGS. 12 and 13. The strap main body 22C has a first surface 22Cr which faces the subject when the case of the wrist device 10 is fixed to the subject, and a second surface 22Cf which faces opposite to the subject side, that is, opposite to the first surface 22Cr. The first surface 22Cr of the strap main body 22C is provided with a plurality of first protrusions 321, 322. Each of the first protrusions 321, 322 protrudes with a height d from the first surface 22Cr. Each of the first protrusions 321, 322 is a square frustum which has a trapezoidal cross section and which has a square shape with the length of one side (length of the base) being the outer dimension D as viewed in a plan view seen from an axial direction perpendicular to the first surface 22Cr.

In this example, the outer dimension D of the first protrusions 321, 322 represents the length of the base of the trapezoid and is set to D=0.15 mm. The height d (depth d) is set to 0.05 mm. The arrangement pattern including the pitch P of 0.3 mm between the first protrusions 321, 322, the pitch P2 of 0.15 mm as the spacing between the first line and the second line, and the P/D ratio of 2 or the like is similar to that of the arrangement example 1 in the foregoing embodiment and therefore will not be described further in detail.

Since the first protrusions 321, 322 according to the modification provided on the portable electronic device strap are arranged with the outer dimension D of the first protrusions 321, 322 and the ratio P/D of the pitch P and the outer dimension D satisfying 0.03 mm≤D≤1.0 mm and 1<P/D≤3, a proper spacing can be secured between the subject (skin of the wrist) and the first surface 22Cr of the strap main body 22C when the case is fixed to the subject. Also, the provision of such first protrusions 321, 322 can secure proper smoothness between the first protrusions 321, 322 (strap main body 22C) and the subject and thus can improve the feeling of wearing. Moreover, the outer dimension D satisfying 0.03 mm D≤1.0 mm can secure a proper size and strength of the first protrusions 321, 322, thus making the first protrusions 321, 322 less likely to be separated due to abrasion or friction.

The first protrusions 221, 222, 321, 322 are shown to have the shape of a hemisphere or square frustum but are not limited to these shapes and may have any shape that can form the first protrusions. Also, if the first protrusions 221, 222, 321, 322 are circular as viewed in a plan view seen from an axial direction perpendicular to the first surface 22r, the center of the circle forming the contour of each of the first protrusions 221, 222, 321, 322 may be defined as the center of each of the first protrusions 221, 222, 321, 322. If the first protrusions 221, 222, 321, 322 are polygonal (particularly with an even number of vertices), the point of intersection of diagonal lines may be defined as the center.

The second protrusions 270, 271 are not limited to the hemispheric shape, either. The second protrusions 270, 271 may have any shape that can form the second protrusions.

The portable electronic device is not limited to the wrist device worn on the wrist of the user as described above and may be a wearable device worn at other parts of the user such as the neck or ankle.

The entire disclosure of Japanese Patent Application No. 2017-137779, filed Jul. 14, 2017 is expressly incorporated by reference herein.

What is claimed is:

1. A portable electronic device comprising:
a sensor which detects at least one of biological information and motion information of a subject;
a case which accommodates the sensor; and
a strap which fixes the case to the subject,
wherein the strap has a first surface which faces the subject when the case is fixed to the subject,
the first surface is provided with a plurality of first protrusions, and
0.03≤mm D≤1.0 mm and 1<P/D≤3 are satisfied, where P/D is a ratio of a pitch P representing a distance between centers of two of the first protrusions and an outer dimension D of each of the first protrusions.

2. The portable electronic device according to claim 1, wherein
the first protrusions are arranged, satisfying 0.03 mm≤D≤0.8 mm and 1.4<P/D≤3.

3. The portable electronic device according to claim 1, wherein
0.005 mm≤d≤0.2 mm is satisfied, where a distance from a top part of the first protrusions to the first surface is defined as a depth d.

4. The portable electronic device according to claim 1, wherein
the pitch P is 0.005 mm≤P≤0.3 mm.

5. The portable electronic device according to claim 1, wherein
the first protrusions have at least
a first line in which the first protrusions are arranged in a first direction, and
a second line in which the first protrusions are arranged in the first direction, with a predetermined spacing from the first line in a second direction orthogonal to the first direction, and
the first protrusions in the first line and the second line are arranged with a shift half the pitch P in the first direction.

6. The portable electronic device according to claim 1, wherein
the strap includes a case fitting section and a strap main body extending from the case fitting section, and
the first protrusions are provided on the strap main body.

7. The portable electronic device according to claim 1, wherein
the strap has a second surface which faces opposite to the subject side when the case is fixed to the subject,
the second surface is provided with a plurality of second protrusions, and
the second protrusions have a pitch P2 and an outer dimension D2 that are different from those of the first protrusions.

8. The portable electronic device according to claim 7, wherein
the first surface and the second surface are made up of different materials from each other.

9. A portable electronic device strap which fixes a case to a subject, the case accommodating a sensor which detects at least one of biological information and motion information of the subject, the portable electronic device strap comprising:
a case fitting section to be fixed to the case;
a strap main body extending from the case fitting section; and
a first surface which faces the subject of the strap main body when the case is fixed to the subject,
wherein the first surface is provided with a plurality of first protrusions, and
0.03 mm≤D≤1.0 mm and 1<P/D≤3 are satisfied, where P/D is a ratio of a pitch P representing a distance between centers of two of the first protrusions and an outer dimension D of each of the first protrusions.

10. The portable electronic device strap according to claim 9, wherein
the first protrusions are arranged, satisfying 0.03 mm≤D≤0.8 mm and 1.4<P/D≤3.

11. The portable electronic device strap according to claim 9, wherein
0.005 mm≤d≤0.2 mm is satisfied, where a distance from a top part of the first protrusions to the first surface is defined as a depth d.

12. The portable electronic device strap according to claim 9, wherein
the pitch P is 0.005 mm≤P≤0.3 mm.

13. The portable electronic device strap according to claim 9, wherein
the first protrusions have at least
a first line in which the first protrusions are arranged in a first direction, and
a second line in which the first protrusions are arranged in the first direction, with a predetermined spacing from the first line in a second direction orthogonal to the first direction, and
the first protrusions in the first line and the second line are arranged with a shift half the pitch P in the first direction.

14. The portable electronic device strap according to claim 9, wherein
the first protrusions are provided on the strap main body.

15. The portable electronic device strap according to claim 9, further comprising
a second surface which faces opposite to the subject side when the case is fixed to the subject,
wherein the second surface is provided with a plurality of second protrusions, and
the second protrusions have a pitch P2 and an outer dimension D2 that are different from those of the first protrusions.

16. The portable electronic device strap according to claim 15, wherein
the first surface and the second surface are made up of different materials from each other.
